(19) United States
(12) Patent Application Publication
(10) Pub. No.: US 2003/0143332 A1
(43) Pub. Date: Jul. 31, 2003

(54) Coating solution for forming insulating film

(75) Inventors: Yuji Yoshida, Edogawa-ku (JP); Nobutaka Kunimi, Tsukuba-shi (JP)

Correspondence Address: SUGHRUE MION, PLLC, 2100 PENNSYLVANIA AVENUE, N.W., WASHINGTON, DC, 20037, US

(73) Assignee: SUMITOMO CHEMICAL COMPANY, LIMITED

(21) Appl. No.: 10/341,410

(22) Filed: Jan. 14, 2003

(30) Foreign Application Priority Data
Jan. 31, 2002 (JP) 2002-023205

(51) Int. Cl. B05D 3/02; C07C 43/20

(52) U.S. Cl. 427/372.2; 568/631

(57) ABSTRACT

An object of the present invention is to provide a coating solution which is capable of forming an insulating film exhibiting a low dielectric constant and superior insulating performance. The object is achieved by a coating solution for forming insulating film comprising at least one selected from the group consisting of a compound represented by the formula (1) and a resin resulting from polymerization of the compound of the formula (1).

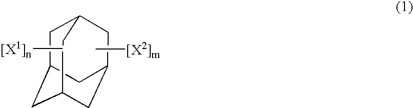

### DETAILED DISCRIPTION OF THE INVENTION

1. Field of the Invention

The present invention relates to coating solution for forming insulating film.

2. Background of the Invention

With finer wiring required for semiconductor devices in recent years, there has arisen a problem of the so-called “wiring delay”, which is a phenomenon that the electronic signal transfer rate is lowered. In order to resolve the wiring delay problem, a method of improving the performance of wiring itself, or a method of reducing the interference between wiring lines are suggested. One approach to reduce the interference between wiring lines is to improve the performance of an insulating film used. To improve the insulating film performance, development of an insulating film having a lowered dielectric constant has been desired.

As it is known that the dielectric constant of a substance is proportional to the electronic polarizability of the substance, attention has been focused on organic materials having a low electronic polarizability. Benzocyclobutene polymer is known as one of organic materials having a low electronic polarizability, however, since this polymer has a dielectric constant of 2.6, it does not ensure sufficient insulating performance. Thus, it has been desired to develop a coating solution capable of forming an insulating film exhibiting superior insulating performance.

### SUMMARY OF THE INVETION

An object of the present invention is to provide a coating solution which is capable of forming an insulating film exhibiting a low dielectric constant and superior insulating performance.

Intensive study has been repeatedly made by the inventors of the present invention to find a coating solution for forming insulating film free of the aforementioned problem and, as a result, the inventors have found that a coating solution comprising at least one selected from the group consisting of adamantane derivatives and resins resulting from polymerization of the respective adamantane derivatives is capable of forming an insulating film having a lowered dielectric constant, and have completed the present invention.

That is, the present invention is directed to a coating solution for forming insulating film comprising at least one selected from the group consisting of: a compound represented by the formula (1):

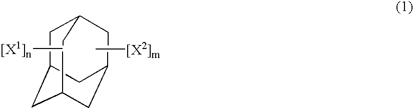

wherein X¹ are the same or different and each represent an alkenyl group having 2 to 6 carbon atoms, an alkynyl group having 2 to 6 carbon atoms, a monovalent organic group represented by the formula (2) below, or a monovalent organic group represented by the formula (3) below; X² may be the same or different when X² is plural and X² represents a hydrogen atom, a halogen atom, a hydroxyl group, an alkyl group having 1 to 6 carbon atoms, an alkoxy group having 1 to 6 carbon atoms, a phenoxy group, or an aryl group which may be substituted; n represents an integer from 2 to 16; and m=16−n; and a resin resulting from polymerization of the compound of the formula (1),

the formula (2) being:

—Y¹—Ar¹ (2)

wherein Y¹ represents an alkenylene group having 2 to 6 carbon atoms or an alkynylene group having 2 to 6 carbon atoms, and Ar¹ represents an aryl group which may be substituted,

the formula (3) being:

—Y²—Ar²—(Y³-A)ₚ (3)

wherein Y² represents a direct bond, an alkylene group having 1 to 6 carbon atoms, or an alkenylene group having 2 to 6 carbon atoms; Y³ represents an alkylene group having 1 to 6 carbon atoms, an alkenylene group having 2 to 6 carbon atoms, or an alkynylene group having 2 to 6 carbon atoms; one of Y² and Y³ is an alkenylene group having 2 to 6 carbon atoms or an alkynylene group having 2 to 6 carbon atoms; p is an integer from 1 to 5; Ar² represents an arylene group which may be substituted; A represents a hydrogen atom or a group equivalent to Ar¹ and A may be the same or different when p is 2 or more.

### DETAILED DESCRIPTION OF THE INVENTION

The coating solution for forming insulating film of the present invention comprises at least one selected from the group consisting of a compound represented by the above formula (1) and a resin resulting from polymerization of the compound represented by the formula (1).

Here, X¹ is the same or different and each represent an alkenyl group having 2 to 6 carbon atoms, an alkynyl group having 2 to 6 carbon atoms, a monovalent organic group represented by the formula (2), or a monovalent organic group represented by the formula (3).

X¹ is preferably an alkynyl group having 2 to 6 carbon atoms, a monovalent organic group represented by the formula (2), or a monovalent organic group represented by the formula (3).

Examples of alkenyl groups having 2 to 6 carbon atoms include a vinyl group, allyl group, propenyl group, butenyl group, butadiynyl group, and hexenyl group. There is no particular limitation on the position of the double bond.

Examples of alkynyl groups having 2 to 6 carbon atoms include an ethynyl group, propynyl group, butynyl group, and hexynyl group. There is no particular limitation on the position of the triple bond.

Examples of Y¹ in the monovalent organic group represented by the formula (2) include alkenylene groups having 2 to 6 carbon atoms such as a vinylene group, propenylene group and butenylene group; or alkynylene groups having 2 to 6 carbon atoms such as an ethynylene group, propynylene group, butynylene group or butadiynylene group.

Ar¹ represents an aryl group which may be substituted. Specific examples of such aryl groups include a phenyl group, methylphenyl group, dimethylphenyl group, ethylphenyl group, diethylphenyl group, trimethylphenyl group, tetramethylphenyl group, pentamethylphenyl group, hydroxyphenyl group, methoxyphenyl group, ethoxyphenyl group, phenoxyphenyl group, fluorophenyl group, chlorophenyl group, bromophenyl group, iodophenyl group, nitrophenyl group, cyanophenyl group, carboxyphenyl group, methyloxycarbonylphenyl group, aminophenyl group, naphthyl group, methylnaphthyl group, dimethylnaphthyl group, ethylnaphthyl group, diethylnaphthyl group, trimethylnaphthyl group, tetramethylnaphthyl group, pentamethylnaphtyl group, hydroxynaphthyl group, methoxynaphthyl group, ethoxynaphthyl group, phenoxynaphthyl group, fluoronaphthyl group, chloronaphthyl group, bromonaphthyl group, iodonaphthyl group, nitronaphthyl group, cyanonaphthyl group, carboxynaphthyl group, methyloxycarbonylnaphthyl group, aminonaphthyl group, biphenyl group, and anthracenyl group.

Y² in the monovalent organic group represented by the formula (3) represents a direct bond, an alkylene group having 1 to 6 carbon atoms, an alkenylene group having 2 to 6 carbon atoms, or an alkynylene group having 2 to 6 carbon atoms. Examples of such alkylene groups having 1 to 6 carbon atoms include a methylene group, ethylene group, propylene group, and hexylene group.

Examples of such alkenylene group having 2 to 6 carbon atoms and examples of such alkynylene groups having 2 to 6 carbon atoms include the same groups as mentioned above.

Y³ represents an alkenyl group having 2 to 6 carbon atoms or an alkynyl group having 2 to 6 carbon atoms, and p is an integer from 1 to 5, preferably 1 or 2.

Examples of such alkenyl groups having 2 to 6 carbon atoms and examples of such alkynyl groups having 2 to 6 carbon atoms include the same groups as mentioned above.

Ar² represents an arylene group which may be substituted. Specific examples of such arylene groups include alkylphenylene groups such as a phenylene group, methylphenylene group, dimethylphenylene group, ethylphenylene group, diethylphenylene group, trimethylphenylene group, tetramethylphenylene group, and pentamethylphenylene group; alkoxyphenylene groups such as a methoxyphenylene group and ethoxyphenylene group; halophenylene groups such as a fluorophenylene group, chlorophenylene group, bromophenylene group, and iodophenylene group; alkylnaphthylene groups such as a methylnaphthylene group, dimethylnaphthylene group, ethylnaphthylene group, diethylnaphthylene group, trimethylnaphthylene group, tetramethylnaphthylene group, and pentamethylnaphtylene group; alkoxynaphthylene groups such as a methoxynaphthylene group and ethoxynaphthylene group; halonaphthylene groups such as a fluoronaphthylene group, chloronaphthylene group, bromonaphthylene group, and iodonaphthylene group; a hydroxyphenylene group, phenoxyphenylene group, nitrophenylene group, cyanophenylene group, carboxyphenylene group, methyloxycarbonylphenylene group, aminophenylene group, naphthylene group, hydroxynaphthylene group, phenoxynaphthylene group, nitronaphthylene group, cyanonaphthylene group, carboxynaphthylene group, methyloxycarbonylnaphthylene group, aminonaphthylene group, biphenylene group, and anthracelene group.

A represents a hydrogen atom or a group equivalent to Ar¹.

X¹ preferably contains a carbon-carbon triple bond because it has high reactivity in polymerization. Preferably, X¹ is an alkynyl group having 2 to 6 carbon atoms, a monovalent organic group of the formula (2) where Y¹ is an alkynylene group having 2 to 6 carbon atoms, or a monovalent organic group represented by above formula (3) where one of Y² and Y³ is an alkynylene group having 2 to 6 carbon atoms.

More preferably, X¹ is a monovalent organic group selected from the group shown below. This case is more preferable because alkynylene groups in the compound can react with each other to form a chemical structure comprising an aromatic ring, a polyvinylene skeleton or a polyacetylene skeleton and, hence, a resulting insulating film will exhibit an enhanced mechanical strength.

—C≡CH

—C≡C—Ar¹ —Ar²(C≡CA)ₚ —C≡C—Ar²(C≡CA)ₚ

wherein Ar¹, Ar² and A represent the same groups as defined in the formulae (2) and (3), and p has the same meaning as in the formula (3).

Much more preferably, X¹ is a monovalent organic group selected from the group shown below. This case is much more preferable because the resulting coating solution exhibits low polarizability and hence is capable of forming an insulating film having a lowered dielectric constant.

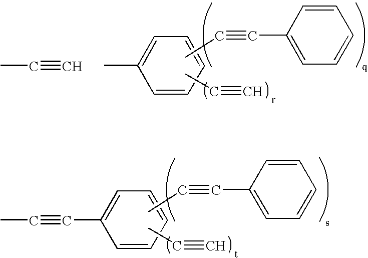

wherein q, r, s and t each represent an integer of from 0 to 5; q+r is from 1 to 5; and s+t is from 0 to 5.

Particularly preferably, X¹ is a monovalent or bivalent organic group selected from the group noted below.

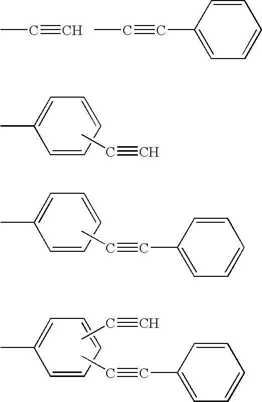

This case is particularly preferable because acetylene, ethynylbenzene, diphenylacetylene and ethynyldiphenylacetylene as raw materials for the following monovalent or bivalent organic groups can be procured easily.

X² represents a hydrogen atom, a halogen atom, a hydroxyl group, an alkyl group having 1 to 6 carbon atoms, an alkoxy group having 1 to 6 carbon atoms, a phenoxy group, or an aryl group which may be substituted, and X²'s may be the same or different.

Examples of such halogen atoms include a fluorine atom, chlorine atom, bromine atom, and iodine atom.

Examples of such alkyl groups having 1 to 6 carbon atoms include a methyl group, ethyl group, propyl group, butyl group, and hexyl group.

Examples of such alkoxy groups having 1 to 6 carbon atoms include a methoxy group, ethoxy group, propoxy group, butoxy group, and hexoxy group.

Examples of such aryl groups which may be substituted include alkylphenyl groups such as a methylphenyl group, dimethylphenyl group, ethylphenyl group, diethylphenyl group, trimethylphenyl group, tetramethylphenyl group, and pentamethylphenyl group; alkoxyphenyl groups such as a methoxyphenyl group and ethoxyphenyl group; halophenyl groups such as a fluorophenyl group, chlorophenyl group, bromophenyl group, and iodophenyl group; alkylnaphthyl groups such as a methylnaphthyl group, dimethylnaphthyl group, ethylnaphthyl group, diethylnaphthyl group, trimethylnaphthyl group, tetramethylnaphthyl group, and pentamethylnaphtyl group; alkoxynaphthyl groups such as a methoxynaphthyl group and ethoxynaphthyl group; halonaphthyl groups such as a fluoronaphthyl group, chloronaphthyl group, bromonaphthyl group, and iodonaphthyl group; a phenyl group, hydroxyphenyl group, phenoxyphenyl group, nitrophenyl group, cyanophenyl group, carboxyphenyl group, methyloxycarbonylphenyl group, aminophenyl group, naphthyl group, hydroxynaphthyl group, phenoxynaphthyl group, nitronaphthyl group, cyanonaphthyl group, carboxynaphthyl group, methyloxycarbonylnaphthyl group, aminonaphthyl group, biphenyl group, and anthracenyl group.

Preferably, X² is a hydrogen atom, a hydroxyl group, or an aryl group which may be substitute; more preferably, X² is a hydrogen atom.

In the formula (1), n represents an integer from 2 to 16 and m=16−n.

Among compounds represented by the formula (1), those compounds having X² as a substituent at the methylene group of adamantane can be prepared by, for example, oxidizing the methylene group of adamantane with a strong acid such as sulfuric acid, nitric acid or fuming sulfuric acid into a carbonyl group and then hydrogenate the carbonyl group to give a compound having a hydroxyl group as X². By further subjecting this hydroxyl group to halogenation using chlorine, bromine, iodine or a like halogen, a compound (1) having a halogen atom as X² can be prepared. By allowing this halogen atom to react with alkyl lithium having 1 to 6 carbon atoms, aryl lithium, alcohol having 1 to 6 carbon atoms or phenol, which are reactive with the halogen atom, a compound having an alkyl group, aryl group, alkoxy group or phenoxy group as X² can be prepared.

Among compounds represented by the formula (1), those compounds having X¹ as a substituent at the methylene group of adamantane can be prepared by, for example, a process including: oxidizing the methylene group of adamantane with a strong acid such as sulfuric acid, nitric acid or fuming sulfuric acid; halogenating the oxidized methylene group with chlorine, bromine, iodine or a like halogen; and allowing the resulting halogen atom to react with a hydrogen atom bonded to an unsaturated group of an alkene having 2 to 6 carbon atoms such as ethylene, propylene or butylene or of an alkyne having 2 to 6 carbon atoms such as acetylene or propynyne or with a hydrogen atom bonded directly to Y¹, Ar¹, Y² or Ar² in the groups represented by the respective formulae (2) and (3), the hydrogen atom being activated with lithium or the like.

Among compounds represented by the formula (1), those compounds having substituents X¹ and/or X² at the bridging methyne group of adamantane can be prepared by, for example, halogenating the bridging methyne group of adamantane with chlorine, bromine, iodine or a like halogen and then subject the halogenated methyne group to a coupling reaction with X¹—H and/or X²—H. The hydrogen atoms of X¹—H and/or X²—H may be activated with metal ion such as lithium, aluminum, titanium or antimony.

In the aforementioned coupling reaction included in the process for preparing the compound having substituent X¹ at the bridging methyne group of adamantane, it is preferable to use a Lewis acid catalyst such as aluminum chloride, tin chloride, antimony chloride, titanium chloride, aluminum bromide, tin bromide, antimony bromide, or titanium bromide. It is more preferable to use t-butyl chloride, t-butyl bromide, t-butyl iodide or the like for coexistence with the catalyst.

Since adamantane as an adamantane derivative to be used as the starting material can be industrially procured with ease and since the methyne group of an adamantane molecule is highly reactive, the compound of the formula (1) is preferably one of the following compounds. The number of X¹ groups per adamantane molecule is preferably two or three because a compound having two or three X¹ groups can be prepared simply.

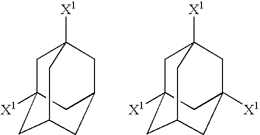

The coating solution for forming insulating film of the present invention can be obtained by dissolving a compound of the formula (1), a resin resulting from polymerization of the compound, or a mixture thereof in an organic solvent.

Known polymerization processes are applicable as the method of polymerizing the compound of the formula (1). Examples of such known processes include: a radical polymerization process using a radical polymerization initiator such as benzoyl peroxide, t-butyl peroxide, or azobisisobutyronitrile; a cation polymerization process using a catalyst such as sulfuric acid, phosphoric acid, triethyl aluminum, or tungsten chloride; an anion polymerization process using a catalyst such as lithium naphthalene; and a photo radical polymerization by irradiation with light or the like.

Usually, the polymerization of the compound of the formula (1) proceeds as X¹ react with each other. Specific examples of resulting resins include poly(diethynyladamantane), poly(triethynyladamantane), poly(tetraethynyladamantane), poly[bis(ethynylphenyl)adamantine], poly[tris(ethynylphenyl)adamantine], poly[bis(diethynylphenyl)adamantine], poly[tris(diethynylphenyl)adamantine], poly[bis(ethynylphenylethynyl)adamantine], and poly[tris(ethynylphenylethynyl)adamantine].

There is no particular limitation on the organic solvent to be used, and, from the viewpoint of high industrial availability and safety, solvents include, for example, alcohol solvents such as methanol, ethanol, isopropanol, 1-butanol, 2-butanol, 1-hexanol, 2-ethoxymethanol, and 3-methoxypropanol; ketone solvents such as acetylacetone, methyl ethyl ketone, methyl isobutyl ketone, 2-pentanone, 3-pentanone, 2-heptanone, and 3-heptanone; ester solvents such as ethyl acetate, propyl acetate, butyl acetate, isobutyl acetate, pentyl acetate, ethyl propionate, propyl propionate, butyl propionate, isobutyl propionate, propylene glycol monomethyl ether acetate, methyl lactate, ethyl lactate, and γ-butyrolactone; ether solvents such as diisopropyl ether, dibutyl ether, ethylpropyl ether, anisole, phenetole, and veratrole; and aromatic hydrocarbon solvents such as mesitylene, ethylbenzene, diethylbenzene, and propylbenzene. These solvents may be used either alone or as a mixture of at least two of them.

The coating solution for forming insulating film of the present invention may further comprise additives such as a radical generator, nonionic surfactant, fluoro-type nonionic surfactant, or silane coupling agent unless the reactivity of the compound of the formula (1) and the performance of the coating solution such as the coating property are impaired.

Examples of such radical generators include t-butyl peroxide, pentyl peroxide, hexyl peroxide, lauroyl peroxide, benzoyl peroxide, and azobisisobutyronitrile.

Examples of such nonionic surfactants include octylpolyethylene oxide, decylpolyethylene oxide, dodecylpolyethylene oxide, octylpolypropylene oxide, decylpolypropylene oxide, and dodecylpolypropylene oxide.

Examples of such fluoro-type nonionic surfactants include perfluorooctylpolyethylene oxide, perfluorodecylpolyethylene oxide, and perfluorododecylpolyethylene oxide.

Examples of such silane coupling agents include vinyltrimethoxysilane, allyltrimethoxysilane, vinyltriethoxysilane, allyltriethoxylsilane, divinyldiethoxylsilane, and trivinylethoxylsilane.

An insulating film can be formed by coating a substrate with the coating solution for forming insulating film of the present invention through any coating process such as spin coating, roller coating, dip coating or scanning and then removing the solvent by a heat treatment. There is no particular limitation on the heating process, and conventional heating processes include a hotplate heating process, a process using a furnace, and a light-irradiation heating process using a xenon lamp performed by an RTP (Rapid Thermal Processor) or the like.

The heat treatment causes X¹ to be coupled to each other to form a three-dimensional structure, which can form an insulating film having excellent mechanical strength and heat resistance. The temperature of the heat treatment is preferably from 200 to 450° C., more preferably from 250 to 400° C., while the heating duration is usually from 1 min to 10 hrs.

The insulating film thus obtained preferably has a dielectric constant of not more than 2.5 and is useful as an insulating film in high-speed operation devices.

The insulating film may be a porous film formed by adding a foaming agent to the coating solution.

### EXAMPLES

Hereinafter, the present invention will be described more specifically by way of examples, which are not intended to limit the scope of the present invention.

### Synthesis Example 1

A 300 mL four-necked flask was charged with 2.72 g (20 mmol) of adamantane, 55.3 g (400 mmol) of t-butyl bromide and 14.2 g (80 mmol) of diphenylacetylene, followed by stirring at room temperature for dissolution. Subsequently, 0.53 g of anhydrous aluminum chloride was added to the resulting solution by portions in one hour. After stirring at room temperature for one hour, the internal temperature of the flask was raised to 50° C. to allow reaction to proceed for one hour. After cooling, the reaction mixture was diluted with 200 g of methylene chloride and then poured into 200 g of ice water containing 20 ml of concentrated hydrochloric acid dissolved therein. After separation of a hydrochloric acid phase, a methylene chloride phase was washed with a saturated saline solution and then with water. The methylene chloride phase was concentrated to 20 g and then poured into 200 g of methanol. Precipitated crystal was separated by filtration and then dried under reduced pressure at 50° C. for 8 hours to give 4.67 g of bis(phenylethynylphenyl)adamantane, which in turn was dissolved in anisole so that the solid content was 10%. The solution thus obtained is herein referred to as “solution a”.

### Synthesis Example 2

A 200 mL four-necked flask was charged with 5.0 g (17 mmol) of dibromoadamantane, 2.3 g (9 mmol) of aluminum bromide and 100 mL of m-dibromobenzene, followed by stirring at 60° C. for 10 hours. After cooling, the reaction mixture was poured into 150 g of ice water containing 10 g of concentrated hydrochloric acid dissolved therein. After stirring, a water phase was removed away. After removal of excess dibromobenzene by distillation under reduced pressure, the resulting residue was added with and dissolved in 100 mL of methylene chloride and the resulting solution was washed with water and a saline solution and then dried over magnesium sulfate as drying agent. After removal of the drying agent by filtration, methylene chloride was concentrated by means of an evaporator and 100 mL of methanol was added to the concentrate, followed by stirring. Precipitated crystal was separated by filtration and then dried under reduced pressure. A 20 mL four-necked flask was charged with 6.0 g of the crystal thus obtained, to which were then added 200 mg of dichlorobis(triphenylphosphine)palladium, 400 mg of triphenylphosphine, 180 mg of copper (I) iodide, and 100 mL of triethylamine. Then, the temperature of the resulting mixture was raised to 70-80° C. Trimethylsilylacetylene in an amount of 6.7 g was added dropwise to the mixture in one hour and reaction was allowed to proceed at the same temperature for four hours. After cooling, the solvent was distilled off and 200 mL of diethyl ether was added to the resulting residue, followed by filtering-off of undissolved salt. The resulting filtrate was washed with 1N hydrochloric acid, saturated saline solution and ultrapure water and the resulting ether phase was dried over magnesium sulfate. Subsequently, the drying agent was filtered off, then the ether was distilled off, and the resulting residue was purified with a column (stationary phase: silica gel 60, developer: hexane/methylene chloride). The principal product in an amount of 5.9 g was dissolved in 150 mL of methanol and 100 mL of tetrahydrofuran and the resulting solution was added with 0.5 g of potassium carbonate and stirred at room temperature for four hours. After distilling-off of the solvent under reduced pressure, 200 mL of methylene chloride and 100 mL of 1N hydrochloric acid were added to the resulting residue. After stirring, the resulting hydrochloric acid phase was removed away. The resulting methylene chloride phase was washed with 100 mL of ultrapure water three times, subjected to distillation to remove the solvent from the methylene chloride phase, and then dried under reduced pressure to give 3.2 g of bis(diethynylphenyl)adamantane, which in turn was dissolved in anisole so that the solid content was 10%. The solution thus obtained is herein referred to as “solution b”.

### Synthesis Example 3

Following the same procedure as in Synthesis Example 2, bis(dibromophenyl)adamantane was obtained. A 200 mL four-necked flask was charged with 6.0 g of this crystal thus obtained, to which were then added 200 mg of dichlorobis(triphenylphosphine)palladium, 400 mg of triphenylphosphine, 180 mg of copper (I) iodide, and 100 mL of triethylamine. Then, the temperature of the resulting mixture was raised to 70-80° C. Ethynylbenzene in an amount of 8.0 g was added dropwise to the mixture in one hour and reaction was allowed to proceed at the same temperature for four hours. After cooling, the solvent was distilled off and 200 mL of diethyl ether was added to the resulting residue, followed by filtering-off of undissolved salt. The resulting filtrate was washed with 1N hydrochloric acid, saturated saline solution and ultrapure water, and the resulting ether phase was dried over magnesium sulfate as drying agent. The drying agent was filtered off, then the ether was distilled off, and the resulting residue was purified with a column (stationary phase: silica gel 60, developer: hexane/methylene chloride). Methanol in an amount of 200 mL was added to the principal product and the resulting solution was stirred and then filtered to obtain precipitated crystal. The crystal thus obtained was dried under reduced pressure to give 5.8 g of bis[(diphenylethynyl)phenyl]adamantane, which in turn was dissolved in anisole so that the solid content was 10%. The solution thus obtained is herein referred to as “solution c”.

### Synthesis Example 4

Following the same procedure as in Sysnthesis Example 2 except that 1-bromobiphenyl was used instead of m-dibromobenzene, 0.7 g of bis(ethynylbiphenyl)adamantane was obtained. Bis(ethynylbiphenyl)adamantane thus obtained was dissolved in anisole so that the solid content was 10%. The solution thus obtained is herein referred to as “solution d”.

### Synthesis Example 5

Following the same procedure as in Synthesis Example 2 except that tribromoadamantane was used instead of dibromoadamantane, 4.5 g of tris(diethynylphenyl)adamantane was obtained. Tris(diethynylphenyl)adamantane thus obtained was dissolved in anisole so that the solid content was 10%. The solution thus obtained is herein referred to as “solution e”.

### Synthesis Example 6

Following the same procedure as in Synthesis Example 3 except that a mixture of 3.3 g of trimethylsilylacetylene and 3.5 g of ethynylbenzene was used instead of trimethylsilylacetylene, 6.9 g of bis[ethynyl(phenylethynyl)phenyl]adamantane was obtained. Bis[ethynyl(phenylethynyl)phenyl]adamantane thus obtained was dissolved in anisole so that the solid content was 10%. The solution thus obtained is herein referred to as “solution f”.

### Synthesis Example 7

According to the method described in the literature reference (A. A. Marik et al., J. Polym. Sci. PART A Polym. Chem., Vol.30, 1747-1757, 1992), triethynyladamantane was obtained. Triethynyladamantane thus obtained was dissolved in anisole so that the solid content was 10% and the resulting solution was stirred at 140-150° C. for 10 hours to give a polytriethynyladamantane solution having a polystyrene-converted weight-average molecular weight of 4200. The solution thus obtained is herein referred to as “resin solution g”.

### Synthesis Example 8

Dibromoadamantane in an amount of 5.8 g (20 mmol), styrene in an amount of 10.2 g (100 mmol), potassium carbonate in an amount of 10 g and 10%-palladium/carbon in an amount of 11.0 g were dissolved in 100 mL of dimethylacetamide and reaction was allowed to proceed at 100° C. for eight hours. After cooling, the reaction solution was filtered through Celite under reduced pressure. To the resulting filtrate was added 250 mL of methylene chloride, and the mixture was washed with 100 mL of 2N hydrochloric acid and further washed with 200 mL ultrapure water three times. The resulting ether phase was dried over magnesium sulfate as drying agent. After removal of the drying agent by filtration, the methylene chloride phase was concentrated to about 40 mL and added dropwise to 500 ml of methanol. Precipitated crystal was separated by filtration and then dried under reduced pressure to give 8.5 g of bisstyryladamantane, which in turn was dissolved in anisole so that the solid content was 10%. The solution thus obtained is herein referred to as “solution h”.

### Examples 1 to 8

The resin solutions in anisole obtained in respective Synthesis Example 1 to 8 were each filtered with a 0.2 μm filter to prepare respective coating solutions.

The coating solutions thus obtained were each applied over a 4-inch silicon wafer by spin coating at 2000 rpm, then prebaked at 150° C. for one minute, and further subjected to a heat treatment in a nitrogen atmosphere under the conditions shown in Table 1. The resulting insulating films were measured for their respective dielectric constants by a mercury probe method (“SSM495” manufactured by S. S. M. Corporation). The results of the measurement are shown in Table 1.

TABLE 1

| | | Heating Conditions | | Dielectric |
|---|---|---|---|---|
| Example | Solutions | Temperature | Duration | Constant |
| 1 | a | 200° C. | 10 min | 2.37 |
| 2 | b | 350° C. | 30 min | 2.80 |
| 3 | c | 350° C. | 30 min | 2.57 |
| 4 | d | 350° C. | 30 min | 2.61 |
| 5 | e | 350° C. | 30 min | 2.48 |
| 6 | f | 400° C. | 30 min | 2.79 |
| 7 | g | 350° C. | 30 min | 2.52 |
| 8 | h | 250° C. | 60 min | 2.60 |

According to the present invention, it is possible to provide an coating solution for forming insulating film which is capable of forming an insulating film having a lowered dielectric constant.

What is claimed is:

1. A coating solution for forming insulating film comprising at least one selected from the group consisting of:
a compound represented by the formula (1): 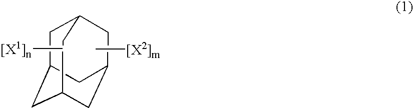
wherein X¹ are the same or different and each represent an alkenyl group having 2 to 6 carbon atoms, an alkynyl group having 2 to 6 carbon atoms, a monovalent organic group represented by the formula (2) below, or a monovalent organic group represented by the formula (3) below; X² may be the same or different when X² is plural and X² represents a hydrogen atom, a halogen atom, a hydroxyl group, an alkyl group having 1 to 6 carbon atoms, an alkoxy group having 1 to 6 carbon atoms, a phenoxy group, or an aryl group which may be substituted; n represents an integer from 2 to 16; and m=16−n; and a resin resulting from polymerization of the compound of the formula (1),
the formula (2) being:
—Y¹—Ar¹ (2)
wherein Y¹ represents an alkenylene group having 2 to 6 carbon atoms or an alkynylene group having 2 to 6 carbon atoms, and Ar¹ represents an aryl group which may be substituted, and
the formula (3) being:
—Y²—Ar²—(Y³-A)ₚ (3)
wherein Y² represents a direct bond, an alkylene group having 1 to 6 carbon atoms, or an alkenylene group having 2 to 6 carbon atoms; Y³ represents an alkylene group having 1 to 6 carbon atoms, an alkenylene group having 2 to 6 carbon atoms, or an alkynylene group having 2 to 6 carbon atoms; one of Y² and Y³ is an alkenylene group having 2 to 6 carbon atoms or an alkynylene group having 2 to 6 carbon atoms; p is an integer from 1 to 5; Ar² represents an arylene group which may be substituted; A represents a hydrogen atom or a group equivalent to Ar¹ and A may be the same or different when p is 2 or more.

2. The coating solution according to claim 1, wherein X¹ is one select from the group consisting of an alkynyl group having 2 to 6 carbon atoms, a monovalent organic group represented by the formula (2), and a monovalent organic group represented by the formula (3).

3. The coating solution according to claim 2, wherein Y¹ of the formula (2) is an alkynylene group having 2 to 6 carbon atoms.

4. The coating solution according to claim 2, wherein at least one of Y² and Y³ of formula (3) is an alkynylene group having 2 to 6 carbon atoms.

5. The coating solution according to claim 1, wherein X¹ is a monovalent organic group selected from the group of
—C≡CH
—C≡C—Ar¹ —Ar²(C≡CA)ₚ —C≡C—Ar²(C≡CA)ₚ

6. The coating solution according to claim 1, wherein X¹ is a monovalent organic group selected from the group of 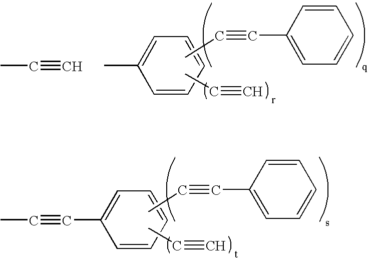
wherein q, r, s and t each represent an integer of from 0 to 5; q+r is from 1 to 5; and s+t is from 0 to 5.

7. The coating solution according to claim 1, wherein X¹ is a monovalent organic group selected from the group of

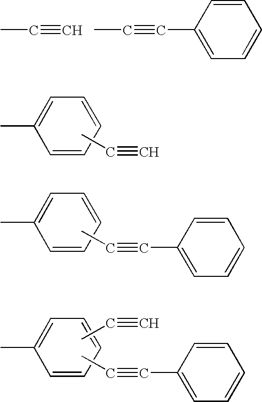

8. The coating solution according to claim 1, wherein the compound represented by the formula (1) is one of the following two compounds.

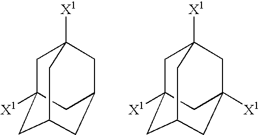

9. A method for forming an insulating film comprising a step of coating the solution according to claim 1 and a step of heat treatment.

10. The method according to claim 9, wherein the step of heat treatment form a three-dimensional structure.
